(19) United States
(12) Patent Application Publication
(10) Pub. No.: US 2026/0124128 A1
(43) Pub. Date: May 7, 2026

(54) COMPOSITION COMPRISING LARGE AMOUNT OF POLYOL

(71) Applicant: L'Oreal, Paris (FR)

(72) Inventors: Takehiko Kasai, Kanagawa (JP); Mitsuru Shimatani, Kanagawa (JP); Shinobu Mitsuda, Kanagawa (JP); Toshifumi Shiroya, Kanagawa (JP); Tomoko Mizuno, Kanagawa (JP); Tatsushi Isojima, Kanagawa (JP); Mariko Yamamoto, Kanagawa (JP)

(73) Assignee: L'Oreal, Paris (FR)

(21) Appl. No.: 19/117,907

(22) PCT Filed: Sep. 15, 2023
(86) PCT No.: PCT/JP2023/034612

§ 371 Date: Apr. 2, 2025

(30) Foreign Application Priority Data
Oct. 4, 2022 (JP) 2022-160348
Nov. 17, 2022 (FR) 2211967

(51) Int. Cl. A61K 8/34; A61K 8/46; A61K 8/55; A61K 8/73; A61K 8/88; A61Q 5/00; A61Q 19/00

(52) U.S. Cl. CPC A61K 8/345; A61K 8/466; A61K 8/55; A61K 8/736; A61K 8/88; A61Q 5/00; A61Q 19/00; A61K 2800/95

(57) ABSTRACT

The present invention relates to a composition comprising: (a) at least one cationic polymer, (b) at least one non-polymeric acid having two or more pKa values or salt(s) thereof; (c) at least one polyol; and (d) water, wherein the amount of the (c) polyol(s) in the composition is 10% by weight or more, relative to the total weight of the composition. The composition according to the present invention can show reduced stickiness although it includes a large amount of polyol such as glycerin.

### TECHNICAL FIELD

The present invention relates to a composition including a relatively large amount of polyol, as well as a cosmetic process using the composition.

### BACKGROUND ART

A polyion complex, which may be in the form of a particle and can be formed with a cationic polymer and an anionic polymer, has already been known.

For example, WO 2021/125069 discloses a composition which is useful for cosmetic treatments and comprises, in one embodiment, at least one polyion complex particle comprising at least one cationic polymer, at least one hyaluronic acid-based anionic polymer and at least one non-polymeric acid having two or more pKa values or a salt thereof. WO 2021/125069 also discloses a specific composition which includes 5% by weight of glycerin.

For example, WO 2022/131351 discloses a composition which is useful for cosmetic treatments and comprises, in one embodiment, at least one polyion complex particle comprising at least one cationic polymer, at least one anionic polymer, at least one non-polymeric acid having two or more pKa values or a salt thereof, at least one filler. WO 2022/131351 also discloses specific compositions which includes 5% by weight of glycerin.

### DISCLOSURE OF INVENTION

Polyol such as glycerin is often used for cosmetic purposes for a keratin substance such as skin. However, a composition including a large amount, i.e., 10% by weight or more relative to the total weight of the composition, of polyol such as glycerin tends to be sticky. Thus, an objective of the present invention is to provide a composition which can show reduced stickiness, while including a large amount, i.e., 10% by weight or more relative to the total weight of the composition, of polyol such as glycerin.

The above objective of the present invention can be achieved by a composition, comprising:
(a) at least one cationic polymer,
(b) at least one non-polymeric acid having two or more pKa values or salt(s) thereof;
(c) at least one polyol; and
(d) water,
wherein
the amount of the (c) polyol(s) in the composition is 10% by weight or more, relative to the total weight of the composition.

The (a) cationic polymer may be crosslinked with the (b) non-polymeric acid having two or more pKa values or salt(s) thereof.

The (a) cationic polymer may have at least one positively chargeable and/or positively charged moiety selected from the group consisting of a primary, secondary or tertiary amino group, a quaternary ammonium group, a guanidine group, a biguanide group, an imidazole group, an imino group, and a pyridyl group.

The (a) cationic polymer may be selected from the group consisting of cyclopolymers of alkyldiallylamine and cyclopolymers of dialkyldiallylammonium such as (co)polydiallyldialkyl ammonium chloride, (co)polyamines such as chitosans and (co)polylysines, cationic (co)polyaminoacids such as collagen, cationic cellulose polymers, and salts thereof.

It may be preferable that the (a) cationic polymer be selected from the group consisting of polylysines, chitosans, and mixtures thereof.

The amount of the (a) cationic polymer(s) in the composition according to the present invention may be from 0.01% to 15% by weight, preferably from 0.05% to 10% by weight, and more preferably from 0.1% to 5% by weight, relative to the total weight of the composition.

The (b) non-polymeric acid having two or more pKa values or salt(s) thereof may be an organic acid or salt(s) thereof, preferably a hydrophilic or water-soluble organic acid or salt(s) thereof, and more preferably phytic acid or salts thereof, terephthalylidene dicamphor sulfonic acid or salts thereof, or a mixture thereof.

The amount of the (b) non-polymeric acid having two or more pKa values or salt(s) thereof in the composition according to the present invention may be from 0.01% to 15% by weight, preferably from 0.05% to 10% by weight, and more preferably from 0.1% to 5% by weight, relative to the total weight of the composition.

It may be preferable that the (c) polyol be glycerin.

The amount of the (c) polyol in the composition according to the present invention may be 95% by weight or less, relative to the total weight of the composition.

The composition according to the present invention may further comprise (e) at least one anionic polymer.

The (e) anionic polymer may be selected from polysaccharides, preferably hyaluronic acid and derivatives thereof, cellulose polymers and salts thereof, and a mixture thereof, and more preferably hyaluronic acid and salts thereof, carboxymethylcellulose and salts thereof, and a mixture thereof.

The amount of the (e) anionic polymer(s) in the composition according to the present invention may be from 0.01% to 15% by weight, preferably from 0.05% to 10% by weight, and more preferably from 0.1% to 5% by weight, relative to the total weight of the composition.

The composition according to the present invention may be a cosmetic composition, preferably a care cosmetic composition, and more preferably a skin care or hair care cosmetic composition.

The present invention also relates to a cosmetic process for keratin substance, comprising applying to the keratin substance the composition according to the present invention; and drying the composition to form a cosmetic film on the keratin substance.

### BEST MODE FOR CARRYING OUT THE INVENTION

After diligent research, the inventors have discovered that it is possible to provide a composition which can show reduced stickiness, while including a large amount, i.e., 10% by weight or more relative to the total weight of the composition, of polyol such as glycerin.

Thus, the composition according to the present invention comprises:
(a) at least one cationic polymer,
(b) at least one non-polymeric acid having two or more pKa values or salt(s) thereof;
(c) at least one polyol; and
(d) water,
wherein
the amount of the (c) polyol(s) in the composition is 10% by weight or more, relative to the total weight of the composition.

The composition according to the present invention can show reduced stickiness although it includes a large amount of polyol such as glycerin.

The stickiness of the composition according to the present invention is lower than that of the composition comprising the ingredient (c) in an amount of 10% by weight or more, relative to the total weight of the composition, without the ingredients (a) and (b).

Thus, the composition according to the present invention can provide improved texture such as smoother feeling to touch.

Polyol such as glycerin is hydrophilic, and therefore, it can be easily washed away with water from the surface of a keratin substance. However, the ingredients (a) and (b) in the composition according to the present invention can form a gel film or coacervate which can maintain polyol well on a keratin substance such as skin.

Thus, the composition according to the present invention can also provide enhanced cosmetic effects derived from polyol, such as enhanced moisturizing effects.

Hereinafter, the composition, process, and the like according to the present invention will be explained in a more detailed manner.

### (Cationic Polymer)

The composition according to the present invention includes (a) at least one cationic polymer.

There is no limit to the type of the (a) cationic polymer. Two or more different types of cationic polymers may be used in combination. Thus, a single type of cationic polymer or a combination of different types of cationic polymers may be used.

A cationic polymer has a positive charge density. The charge density of the (a) cationic polymer may be from 0.01 meq/g to 20 meq/g, preferably from 0.05 to 15 meq/g, and more preferably from 0.1 to 10 meq/g.

It may be preferable that the molecular weight of the (a) cationic polymer be 1,000 or more, preferably 2,000 or more, more preferably 3,000 or more, and even more preferably 4,000 or more.

Unless otherwise defined in the descriptions, “molecular weight” means a number average molecular weight.

The (a) cationic polymer may have at least one positively chargeable and/or positively charged moiety selected from the group consisting of a primary, secondary or tertiary amino group, a quaternary ammonium group, a guanidine group, a biguanide group, an imidazole group, an imino group, and a pyridyl group. The term (primary) “amino group” here means an —NH₂ group.

The (a) cationic polymer may be a homopolymer or a copolymer. The term “copolymer” is understood to mean both copolymers obtained from two kinds of monomers and those obtained from more than two kinds of monomers, such as terpolymers obtained from three kinds of monomers.

The (a) cationic polymer may be selected from natural and synthetic cationic polymers. Non-limiting examples of the (a) cationic polymers are as follows.
(1) Homopolymers and copolymers derived from acrylic or methacrylic esters and amides and comprising at least one unit chosen from units of the following formulas:

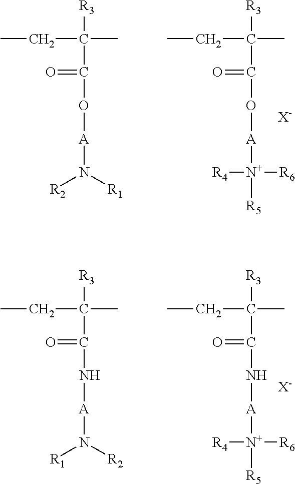

wherein:
R₁ and R₂, which may be identical or different, are chosen from hydrogen and alkyl groups comprising from 1 to 6 carbon atoms, for instance, methyl and ethyl groups;
R₃, which may be identical or different, is chosen from hydrogen and CH₃; the symbols A, which may be identical or different, are chosen from linear or branched alkyl groups comprising from 1 to 6 carbon atoms, for example, from 2 to 3 carbon atoms and hydroxyalkyl groups comprising from 1 to 4 carbon atoms;
R₄, R₅, and R₆, which may be identical or different, are chosen from alkyl groups comprising from 1 to 18 carbon atoms and benzyl groups, and in at least one embodiment, alkyl groups comprising from 1 to 6 carbon atoms; and
X is an anion derived from an inorganic or organic acid, such as methosulphate anions and halides, for instance chloride and bromide.

The copolymers of family (1) above may also comprise at least one unit derived from co-monomers which may be chosen from acrylamides, methacrylamides, diacetone acrylamides, acrylamides and methacrylamides substituted on the nitrogen atom with (C₁-C₄) lower alkyl groups, groups derived from acrylic or methacrylic acids and esters thereof, vinyllactams such as vinylpyrrolidone and vinylcaprolactam, and vinyl esters.

Examples of copolymers of family (1) include, but are not limited to:
copolymers of acrylamide and of dimethylaminoethyl methacrylate quaternized with dimethyl sulphate or with a dimethyl halide,
copolymers of acrylamide and of methacryloyloxyethyltrimethylammonium chloride described, for example, in European Patent Application No. 0 080 976,
copolymers of acrylamide and of methacryloyloxyethyltrimethylammonium methosulphate, quaternized or nonquaternized vinylpyrrolidone/dialkylaminoalkyl acrylate or methacrylate copolymers, described, for example, in French Patent Nos. 2 077 143 and 2 393 573, dimethylaminoethyl methacrylate/vinylcaprolactam/vinylpyrrolidone terpolymers, vinylpyrrolidone/methacrylamidopropyldimethylamine copolymers, quaternized vinylpyrrolidone/dimethylaminopropylmethacrylamide copolymers, and crosslinked methacryloyloxy(C₁-C₄)alkyltri(C₁-C₄)alkylammonium salt polymers such as the polymers obtained by homopolymerization of dimethylaminoethyl methacrylate quatemized with methyl chloride, or by copolymerization of acrylamide with dimethylaminoethyl methacrylate quaternized with methyl chloride, the homopolymerization or copolymerization being followed by crosslinking with a compound containing an olefinic unsaturation, for example, methylenebisacrylamide.
(2) Cationic cellulose polymers such as cellulose ether derivatives comprising one or more quaternary ammonium groups described, for example, in French Patent No. 1 492 597, such as the polymers sold under the names “JR” (JR 400, JR 125, JR 30M) or “LR” (LR 400, LR 30M) by the company Union Carbide Corporation. These polymers are also defined in the CTFA dictionary as quaternary ammoniums of hydroxyethylcellulose that have reacted with an epoxide substituted with a trimethylammonium group.

It is preferable that the cationic cellulose polymer have at least one quaternary ammonium group, preferably a quaternary trialkyl ammonium group, and more preferably a quaternary trimethyl ammonium group.

The quaternary ammonium group may be present in a quaternary ammonium group-containing group which may be represented by the following chemical formula (I):

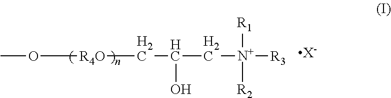

wherein
each of R₁ and R₂ denotes a C₁-C₃ alkyl group, preferably a methyl or ethyl group, and more preferably a methyl group,
R₃ denotes a C₁-C₂₄ alkyl group, preferably a methyl or ethyl group, and more preferably methyl group,
X— denotes an anion, preferably a halide, and more preferably a chloride,
n denotes an integer from 0-30, preferably 0-10, and more preferably 0, and
R₄ denotes a C₁-C₄ alkylene group, preferably an ethylene or propylene group.

The leftmost ether bond (—O—) in the above chemical formula (I) can attach to the sugar ring of the polysaccharide.

It is preferable that the quaternary ammonium group-containing group be —O—CH₂—CH(OH)—CH₂—N⁺ (CH₃)₃.
(3) Cationic cellulose polymers such as cellulose copolymers and cellulose derivatives grafted with a water-soluble monomer of quaternary ammonium, and described, for example, in U.S. Pat. No. 4,131,576, such as hydroxyalkylcelluloses, for instance, hydroxymethyl-, hydroxyethyl-, and hydroxypropylcelluloses grafted, for example, with a salt chosen from methacryloylethyltrimethylammonium, methacrylamidopropyltrimethylammonium, and dimethyldiallylammonium salts.

Commercial products corresponding to these polymers include, for example, the products sold under the name “Celquat® L 200” and “Celquat® H 100” by the company National Starch.
(4) Non-cellulose-based cationic polysaccharides described in U.S. Pat. Nos. 3,589,578 and 4,031,307, such as guar gums comprising cationic trialkylammonium groups, cationic hyaluronic acid, and dextran hydroxypropyl trimonium chloride. Guar gums modified with a salt, for example the chloride, of 2,3-epoxypropyltrimethylammonium (guar hydroxypropyltrimonium chloride) may also be used.

Such products are sold, for instance, under the trade names JAGUAR® C13 S, JAGUAR® C15, JAGUAR® C17, and JAGUAR® C162 by the company MEYHALL.
(5) Polymers comprising piperazinyl units and divalent alkylene or hydroxyalkylene groups comprising straight or branched chains, optionally interrupted with at least one entity chosen from oxygen, sulphur, nitrogen, aromatic rings, and heterocyclic rings, and also the oxidation and/or quaternization products of these polymers. Such polymers are described, for example, in French Patent Nos. 2 162 025 and 2 280 361.
(6) Water-soluble polyamino amides prepared, for example, by polycondensation of an acidic compound with a polyamine; these polyamino amides possibly being crosslinked with an entity chosen from epihalohydrins; diepoxides; dianhydrides; unsaturated dianhydrides; bisunsaturated derivatives; bishalohydrins; bisazetidiniums; bishaloacyidiamines; bisalkyl halides; oligomers resulting from the reaction of a difunctional compound which is reactive with an entity chosen from bishalohydrins, bisazetidiniums, bishaloacyldiamines, bisalkyl halides, epihalohydrins, diepoxides, and bisunsaturated derivatives; the crosslinking agent being used in an amount ranging from 0.025 to 0.35 mol per amine group of the polyamino amide; these polyamino amides optionally being alkylated or, if they comprise at least one tertiary amine function, they may be quaternized. Such polymers are described, for example, in French Patent Nos. 2 252 840 and 2 368 508.
(7) Polyamino amide derivatives resulting from the condensation of polyalkylene polyamines with polycarboxylic acids, followed by alkylation with difunctional agents, for example, adipic acid/dialkylaminohydroxyalkyldialkylenetriamine polymers in which the alkyl group comprises from 1 to 4 carbon atoms, such as methyl, ethyl, and propyl groups, and the alkylene group comprises from 1 to 4 carbon atoms, such as an ethylene group. Such polymers are described, for instance, in French Patent No. 1 583 363. In at least one embodiment, these derivatives may be chosen from adipic acid/dimethylaminohydroxypropyldiethylenetriamine polymers.
(8) Polymers obtained by reaction of a polyalkylene polyamine comprising two primary amine groups and at least one secondary amine group, with a dicarboxylic acid chosen from diglycolic acid and saturated aliphatic dicarboxylic acids comprising from 3 to 8 carbon atoms. The molar ratio of the polyalkylene polyamine to the dicarboxylic acid may range from 0.8:1 to 1.4:1; the polyamino amide resulting therefrom being reacted with epichlorohydrin in a molar ratio of epichlorohydrin relative to the secondary amine group of the polyamino amide ranging from 0.5:1 to 1.8:1. Such polymers are described, for example, in U.S. Pat. Nos. 3,227,615 and 2,961,347.
(9) Cyclopolymers of alkyldiallylamine and cyclopolymers of dialkyldiallyl-ammonium, such as homopolymers and copolymers comprising, as the main constituent of the chain, at least one unit chosen from units of formulas (Ia) and (Ib):

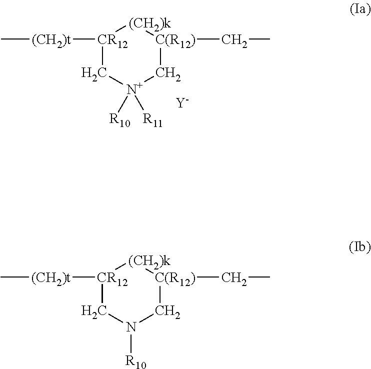

wherein:
k and t, which may be identical or different, are equal to 0 or 1, the sum k+t being equal to 1;
R₁₂ is chosen from hydrogen and methyl groups;
R₁₀ and R₁₁, which may be identical or different, are chosen from alkyl groups comprising from 1 to 6 carbon atoms, hydroxyalkyl groups in which the alkyl group comprises, for example, from 1 to 5 carbon atoms, and lower (C₁-C₄)amidoalkyl groups, or R₁₀ and R₁₁ may form, together with the nitrogen atom to which they are attached, heterocyclic groups such as piperidinyl and morpholinyl; and
Y′ is an anion such as bromide, chloride, acetate, borate, citrate, tartrate, bisulphate, bisulphite, sulphate, and phosphate. These polymers are described, for example, in French Patent No. 2 080 759 and in its Certificate of Addition 2 190 406.

In one embodiment, R₁₀ and R₁₁, which may be identical or different, are chosen from alkyl groups comprising from 1 to 4 carbon atoms.

Examples of such polymers include, but are not limited to, (co)polydiallyldialkyl ammonium chloride such as the dimethyidiallylammonium chloride homopolymer (polyquaternium-6) sold under the name “MERQUAT® 100” by the company CALGON (and its homologues of low weight-average molecular mass) and the copolymers of diallyldimethylammonium chloride and of acrylamide sold under the name “MERQUAT® 550”.

Quaternary diammonium polymers comprising at least one repeating unit of formula (II):

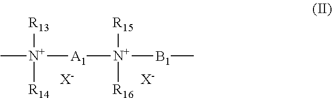

wherein:
R₁₃, R₁₄, R₁₅, and R₁₆, which may be identical or different, are chosen from aliphatic, alicyclic, and arylaliphatic groups comprising from 1 to 20 carbon atoms and lower hydroxyalkyl aliphatic groups, or alternatively R₁₃, R₁₄, R₁₅, and R₁₆ may form, together or separately, with the nitrogen atoms to which they are attached, heterocycles optionally comprising a second heteroatom other than nitrogen, or alternatively R₁₃, R₁₄, R₁₅, and R₁₆, which may be identical or different, are chosen from linear or branched C₁-C₆ alkyl groups substituted with at least one group chosen from nitrile groups, ester groups, acyl groups, amide groups, —CO—O—R₁₇-E groups, and —CO—NH—R₁₇-E groups, wherein R₁₇ is an alkylene group and E is a quaternary ammonium group;
A₁ and B₁, which may be identical or different, are chosen from polymethylene groups comprising from 2 to 20 carbon atoms, which may be linear or branched, saturated or unsaturated, and which may comprise, linked or intercalated in the main chain, at least one entity chosen from aromatic rings, oxygen, sulphur, sulphoxide groups, sulphone groups, disulphide groups, amino groups, alkylamino groups, hydroxyl groups, quaternary ammonium groups, ureido groups, amide groups, and ester groups, and
X⁻ is an anion derived from an inorganic or organic acid;
A₁, R₁₃, and R₁₅ may form, together with the two nitrogen atoms to which they are attached, a piperazine ring;
if A₁ is chosen from linear or branched, saturated or unsaturated alkylene or hydroxyalkylene groups, B₁ may be chosen from:

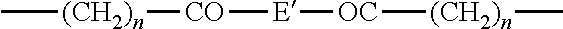

wherein E′ is chosen from:
a) glycol residues of formula —O—Z—O—, wherein Z is chosen from linear or branched hydrocarbon-based groups and groups of the following formulas:

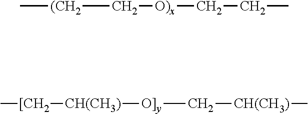

wherein x and y, which may be identical or different, are chosen from integers ranging from 1 to 4, which represent a defined and unique degree of polymerization, and numbers ranging from 1 to 4, which represent an average degree of polymerization;
b) bis-secondary diamine residue such as piperazine derivatives;
c) bis-primary diamine residues of formula —NH—Y—NH—, wherein Y is chosen from linear or branched hydrocarbon-based groups and the divalent group —CH₂—CH₂—S—S—CH₂—CH₂—; and
d) ureylene groups of formula —NH—CO—NH—.

In at least one embodiment, X⁻ is an anion such as chloride or bromide. Polymers of this type are described, for example, in French Patent Nos. 2 320 330; 2 270 846; 2 316 271; 2 336 434; and 2 413 907 and U.S. Pat. Nos. 2,273,780; 2,375,853; 2,388,614; 2,454,547; 3,206,462; 2,261,002; 2,271,378; 3,874,870; 4,001,432; 3,929,990; 3,966,904; 4,005,193; 4,025,617; 4,025,627; 4,025,653; 4,026,945; and 4,027,020. Non-limiting examples of such polymers include those comprising at least one repeating unit of formula (III):

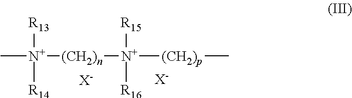

wherein R₁₃, R₁₄, R₁₅, and R₁₆, which may be identical or different, are chosen from alkyl and hydroxyalkyl groups comprising from 1 to 4 carbon atoms, n and p, which may be identical or different, are integers ranging from 2 to 20, and X⁻ is an anion derived from an inorganic or organic acid.
(11) Polyquaternary ammonium polymers comprising units of formula (IV):

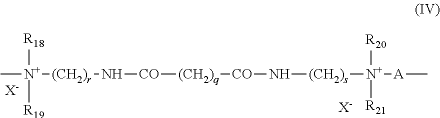

wherein:
R₁₈, R₁₉, R₂₀, and R₂₁, which may be identical or different, are chosen from hydrogen, methyl groups, ethyl groups, propyl groups, p-hydroxyethyl groups, β-hydroxypropyl groups, —CH₂CH₂(OCH₂CH₂)ₚOH groups, wherein p is chosen from integers ranging from 0 to 6, with the proviso that R₁₈, R₁₉, R₂₀, and R₂₁ are not simultaneously hydrogen,
r and s, which may be identical or different, are chosen from integers ranging from 1 to 6,
q is chosen from integers ranging from 0 to 34,
X⁻ is an anion such as a halide, and
A is chosen from radicals of dihalides and —CH₂—CH₂—O—CH₂—CH₂—.

Such compounds are described, for instance, in European Patent Application No. 0 122 324.
(12) Quaternary polymers of vinylpyrrolidone and of vinylimidazole.

Other examples of suitable cationic polymers include, but not limited to, cationic proteins and cationic protein hydrolysates, polyalkyleneimines, such as polyethyleneimines, polymers comprising units chosen from vinylpyridine and vinylpyridinium units, condensates of polyamines and of epichlorohydrin, quaternary polyureylenes, and chitin derivatives.

According to one embodiment of the present invention, the (a) cationic polymer is chosen from cellulose ether derivatives comprising quaternary ammonium groups, such as the products sold under the name “JR 400” by the company UNION CARBIDE CORPORATION, cationic cyclopolymers, for instance, the homo-polymers and copolymers of dimethyldiallylammonium chloride sold under the names MERQUAT® 100, MERQUAT® 550, and MERQUAT® S by the company CALGON, guar gums modified with a 2,3-epoxypropyltrimethylammonium salt, and quaternary polymers of vinylpyrrolidone and of vinylimidazole.
(13) Polyamines

As the (a) cationic polymer, it is also possible to use (co)polyamines, which may be homopolymers or copolymers, with a plurality of amino groups. The amino group may be a primary, secondary, tertiary or quaternary amino group. The amino group may be present in a polymer backbone or a pendent group, if present, of the (co)polyamines.

As an example of the (co)polyamines, mention may be made of chitosan, (co)polyallylamines, (co)polyvinylamines, (co)polyanilines, (co)polyvinylimidazoles, (co)polydimethylaminoethylenemethacrylates, (co)polyvinylpyridines such as (co)poly-1-methyl-2-vinylpyridines, (co)polyimines such as (co) polyethyleneimines, (co)polypyridines such as (co)poly(quaternary pyridines), (co)polybiguanides such as (co)polyaminopropyl biguanides, (co)polylysines, (co)polyomithines, (co)polyarginines, (co)polyhistidines, aminodextrans, aminocelluloses, amino(co)polyvinylacetals, and salts thereof.

It may be preferable that the (a) cationic polymer be selected from chitosans.

It may also be preferable that the (a) cationic polymer be selected from (co)polylysines.

Polylysine is well known. Polylysine can be a natural homopolymer of L-lysine that can be produced by bacterial fermentation. For example, polylysine can be α-Poly-L-lysine and c-Poly-L-lysine, typically used as a natural preservative in food products. Polylysine is a polyelectrolyte which is soluble in polar solvents such as water, propylene glycol and glycerol. Polylysine is commercially available in various forms, such as poly D-lysine and poly L-lysine. Polylysine can be in salt and/or solution form.
(14) Cationic Polyaminoacids

As the (a) cationic polymer, it may be possible use cationic polyaminoacids, which may be cationic homopolymers or copolymers, with a plurality of amino groups and carboxyl groups.

The amino group may be a primary, secondary, tertiary or quaternary amino group. The amino group may be present in a polymer backbone or a pendent group, if present, of the cationic polyaminoacids. The carboxyl group may be present in a pendent group, if present, of the cationic polyaminoacids.

As examples of the cationic polyaminoacids, mention may be made of cationized collagen, cationized gelatin, steardimonium hydroxypropyl hydrolyzed wheat protein, cocodimonium hydroxypropyl hydrolyzed wheat protein, hydroxypropyltrimonium hydrolyzed conchiolin protein, steardimonium hydroxypropyl hydrolyzed soy protein, hydroxypropyltrimonium hydrolyzed soy protein, cocodimonium hydroxypropyl hydrolyzed soy protein, and the like.

The following descriptions relate to preferable embodiments of the (a) cationic polymer.

It may be preferable that the (a) cationic polymer be selected from the group consisting of cyclopolymers of alkyldiallylamine and cyclopolymers of dialkyldiallylammonium such as (co)polydiallyldialkyl ammonium chloride, (co)polyamines such as chitosans and (co)polylysines, cationic (co)polyaminoacids such as cationized collagen, cationic cellulose polymers, and salts thereof.

It may be more preferable that the (a) cationic polymer be selected from the group consisting of polylysines, chitosans, and mixtures thereof.

The amount of the (a) cationic polymer(s) in the composition according to the present invention may be 0.01% by weight or more, preferably 0.05% by weight or more, and more preferably 0.1% by weight or more, relative to the total weight of the composition.

The amount of the (a) cationic polymer(s) in the composition according to the present invention may be 15% by weight or less, preferably 10% by weight or less, and more preferably 5% by weight or less, relative to the total weight of the composition.

The amount of the (a) cationic polymer(s) in the composition according to the present invention may be from 0.01% to 15% by weight, preferably from 0.05% to 10% by weight, and more preferably from 0.1% to 5% by weight, relative to the total weight of the composition.

### (Anionic Polymer)

The composition according to the present invention may include (e) at least one anionic polymer.

If the composition according to the present invention includes the (e) anionic polymer(s), the (a) cationic polymer(s) may form polyion complex(es) with the (e) anionic polymer(s).

There is no limit to the type of the (e) anionic polymer. Two or more different types of anionic polymers may be used in combination. Thus, a single type of anionic polymer or a combination of different types of anionic polymers may be used.

An anionic polymer has a positive charge density. The charge density of the (e) anionic polymer may be from 0.1 meq/g to 20 meq/g, preferably from 1 to 15 meq/g, and more preferably from 4 to 10 meq/g if the (e) anionic polymer is a synthetic anionic polymer, and the average substitution degree of the anionic polymer may be from 0.1 to 3.0, preferably from 0.2 to 2.7, and more preferably from 0.3 to 2.5 if the (e) anionic polymer is a natural anionic polymer.

It may be preferable that the molecular weight of the (e) anionic polymer be 1,000 or more, preferably 3,000 or more, even more preferably 5,000 or more, even more preferably 10,000 or more, even more preferably 50,000 or more, even more preferably 100,000 or more, and even more preferably 1,000,000 or more.

Unless otherwise defined in the descriptions, “molecular weight” may mean a number average molecular weight.

The (e) anionic polymer may have at least one negatively chargeable and/or negatively charged moiety selected from the group consisting of a sulfuric group, a sulfate group, a sulfonic group, a sulfonate group, a phosphoric group, a phosphate group, a phosphonic group, a phosphonate group, a carboxylic group, and a carboxylate group.

The (e) anionic polymer may be a homopolymer or a copolymer. The term “copolymer” is understood to mean both copolymers obtained from two kinds of monomers and those obtained from more than two kinds of monomers, such as terpolymers obtained from three kinds of monomers.

The (e) anionic polymer may be selected from natural and synthetic anionic polymers.

The (e) anionic polymer may comprise at least one hydrophobic chain.

The anionic polymer, which may comprise at least one hydrophobic chain, may be obtained by copolymerization of a monomer (a) chosen from carboxylic acids comprising an α,β-ethylenic unsaturation (monomer a′) and 2-acrylamido-2-methylpropanesulphonic acid (monomer a″) with a non-surface-active monomer (b) comprising an ethylenic unsaturation other than (a) and/or a monomer (c) comprising an ethylenic unsaturation resulting from the reaction of an acrylic monomer comprising an α,β-monoethylenic unsaturation or of an isocyanate monomer comprising a monoethylenic unsaturation with a monohydric nonionic amphiphilic component or with a primary or secondary fatty amine.

Thus, the anionic polymer with at least one hydrophobic chain may be obtained by two synthetic routes:
either by copolymerization of the monomers (a′) and (c), or (a′), (b) and (c), or (a″) and (c), or (a″), (b) and (c),
or by modification (and in particular esterification or amidation) of a copolymer formed from the monomers (a′) or from the monomers (a′) and (b), or (a″) and (b), by a monohydric nonionic amphiphilic compound or a primary or secondary fatty amine.

Mention may in particular be made, as 2-acrylamido-2-methylpropanesulphonic acid copolymers, of those disclosed in the article “Micelle formation of random copolymers of sodium 2-(acrylamido)-2-methylpropanesulfonate and nonionic surfactant macromonomer in water as studied by fluorescence and dynamic light scattering—Macromolecules, 2000, Vol. 33, No. 10-3694-3704” and in applications EP-A-0 750 899 and EP-A-1 069 172.

The carboxylic acid comprising an α,β-monoethylenic unsaturation constituting the monomer (a′) can be chosen from numerous acids and in particular from acrylic acid, methacrylic acid, crotonic acid, itaconic acid and maleic acid. It is preferably acrylic or methacrylic acid.

The copolymer can comprise a monomer (b) comprising a monoethylenic unsaturation which does not have a surfactant property. The preferred monomers are those which give water-insoluble polymers when they are homopolymerized. They can be chosen, for example, from C₁-C₄ alkyl acrylates and methacrylates, such as methyl acrylate, ethyl acrylate, butyl acrylate or the corresponding methacrylates. The more particularly preferred monomers are methyl acrylate and ethyl acrylate. The other monomers which can be used are, for example, styrene, vinyltoluene, vinyl acetate, acrylonitrile and vinylidene chloride. Unreactive monomers are preferred, these monomers being those in which the single ethylenic group is the only group which is reactive under the polymerization conditions. However, monomers which comprise groups which react under the effect of heat, such as hydroxyethyl acrylate, can optionally be used.

The monomer (c) is obtained by reaction of an acrylic monomer comprising α,β-monoethylenic unsaturation, such as (a), or of an isocyanate monomer comprising monoethylenic unsaturation with a monohydric nonionic amphiphilic compound or a primary or secondary fatty amine.

The monohydric nonionic amphiphilic compounds or the primary or secondary fatty amines used to produce the nonionic monomer (c) are well known. The monohydric nonionic amphiphilic compounds are generally alkoxylated hydrophobic compounds comprising an alkylene oxide forming the hydrophilic part of the molecule. The hydrophobic compounds are generally composed of an aliphatic alcohol or an alkylphenol, in which compounds a carbonaceous chain comprising at least six carbon atoms constitutes the hydrophobic part of the amphiphilic compound.

The preferred monohydric nonionic amphiphilic compounds are compounds having the following formula (V):

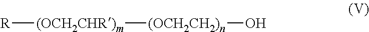

in which R is chosen from alkyl or alkylene groups comprising from 6 to 30 carbon atoms and 50 alkylaryl groups having alkyl radicals comprising from 8 to 30 carbon atoms, R′ is chosen from alkyl groups comprising from 1 to 4 carbon atoms, n is a mean number ranging from approximately 1 to 150 and m is a mean number ranging from approximately 0 to 50, provided that n is at least as great as m.

Preferably, in the compounds of formula (V), the R group is chosen from alkyl groups comprising from 12 to 26 carbon atoms and alkylphenyl groups in which the alkyl group is C₈-C₁₃; the R′ group is the methyl group; m=0 and n=1 to 25.

The preferred primary and secondary fatty amines are composed of one or two alkyl chains comprising from 6 to 30 carbon atoms.

The monomer used to form the nonionic urethane monomer (c) can be chosen from highly varied compounds. Use may be made of any compound comprising a copolymerizable unsaturation, such as an acrylic, methacrylic or allylic unsaturation. The monomer (c) can be obtained in particular from an isocyanate comprising a monoethylenic unsaturation, such as, in particular, α,α-dimethyl-m-isopropenylbenzyl isocyanate.

The monomer (c) can be chosen in particular from acrylates, methacrylates or itaconates of oxyethylenated (1 to 50 EO) C₆-C₃₀ fatty alcohol, such as steareth-20 methacrylate, oxyethylenated (25 EO) behenyl methacrylate, oxyethylenated (20 EO) monocetyl itaconate, oxyethylenated (20 EO) monostearyl itaconate or the acrylate modified by polyoxyethylenated (25 EO) C₁₂-C₂₄ alcohols and from dimethyl-m-isopropenylbenzyl isocyanates of oxyethylenated (1 to 50 EO) C₆-C₃₀ fatty alcohol, such as, in particular, the dimethyl-m-isopropenylbenzyl isocyanate of oxyethylenated behenyl alcohol.

According to a specific embodiment of the present invention, the (e) anionic polymer is chosen from acrylic terpolymers obtained from (a) a carboxylic acid comprising an α,β-ethylenic unsaturation, (b) a non-surface-active monomer comprising an ethylenic unsaturation other than (a), and (c) a nonionic urethane monomer which is the reaction product of a monohydric nonionic amphiphilic compound with an isocyanate comprising a monoethylenic unsaturation.

Mention may in particular be made, as anionic polymers comprising at least one hydrophobic chain, of the acrylic acid/ethyl acrylate/alkyl acrylate terpolymer, such as the product as a 30% aqueous dispersion sold under the name Acusol 823 by Rohm & Haas; the acrylates/steareth-20 methacrylate copolymer, such as the product sold under the name Aculyn 22 by Rohm & Haas; the (meth)acrylic acid/ethyl acrylate/oxyethylenated (25 EO) behenyl methacrylate terpolymer, such as the product as an aqueous emulsion sold under the name Aculyn 28 by Rohm & Haas; the acrylic acid/oxyethylenated (20 EO) monocetyl itaconate copolymer, such as the product as a 30% aqueous dispersion sold under the name Structure 3001 by National Starch; the acrylic acid/oxyethylenated (20 EO) monostearyl itaconate copolymer, such as the product as a 30% aqueous dispersion sold under the name Structure 2001 by National Starch; the acrylates/acrylate modified by polyoxyethylenated (25 EO) C₁₂-C₂₄ alcohol copolymer, such as the 30-32% copolymer latex sold under the name Synthalen W2000 by 3V SA; or the methacrylic acid/methyl acrylate/dimethyl-meta-isopropenylbenzyl isocyanate of ethoxylated behenyl alcohol terpolymer, such as the product as a 24% aqueous dispersion and comprising 40 ethylene oxide groups disclosed in the document EP-A-0 173 109.

The (e) anionic polymers may also be Polyester-5, such as the product sold under the name of Eastman AQ™ 55S Polymer by EASTMAN CHEMICAL having the chemical formula below.

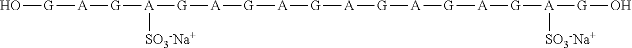

A: dicarboxylic acid moiety
G: glycol moiety
SO₃³¹ Na⁺: sodium sulfo group
OH: hydroxyl group

It may be preferable that the (e) anionic polymer be selected from the group consisting of polysaccharides such as alginic acid, hyaluronic acid, and cellulose polymers (e.g., carboxymethylcellulose and cellulose gum), anionic (co)polyaminoacids such as (co)polyglutamic acids, (co)poly(meth)acrylic acids, (co)polyamic acids, (co)polystyrene sulfonate, (co)poly(vinyl sulfate), dextran sulfate, chondroitin sulfate, (co)polymaleic acids, (co)polyfumaric acids, maleic acid (co)polymers, and salts thereof.

The maleic acid copolymer may comprise one or more maleic acid comonomers, and one or more comonomers chosen from vinyl acetate, vinyl alcohol, vinylpyrrolidone, olefins comprising from 2 to 20 carbon atoms, and styrene.

Thus, the “maleic acid copolymer” is understood to mean any polymer obtained by copolymerization of one or more maleic acid comonomers and of one or more comonomers chosen from vinyl acetate, vinyl alcohol, vinylpyrrolidone, olefins comprising from 2 to 20 carbon atoms, such as octadecene, ethylene, isobutylene, diisobutylene or isooctylene, and styrene, the maleic acid comonomers optionally being partially or completely hydrolysed.

Use will preferably be made of hydrophilic polymers, that is to say polymers having a solubility in water of greater than or equal to 2 g/l.

In an advantageous aspect of the present invention, the maleic acid copolymer may have a molar fraction of maleic acid units of between 0.1 and 1, more preferably between 0.4 and 0.9.

The weight-average molar mass of the maleic acid copolymer may be between 1,000 and 500,000, and preferably between 1,000 and 50,000.

It is preferable that the maleic acid copolymer be a styrene/maleic acid copolymer, and more preferably sodium styrene/maleic acid copolymer.

Use will preferably be made of a copolymer of styrene and of maleic acid in a 50/50 ratio.

Use may be made, for example, of the styrene/maleic acid (50/50) copolymer, in the form of an ammonium salt at 30% in water, sold under the reference SMA1000H® by Cray Valley or the styrene/maleic acid (50/50) copolymer, in the form of a sodium salt at 40% in water, sold under the reference SMAl000HNa® by Cray Valley.

The use of the styrene/maleic acid copolymer such as sodium styrene/maleic acid copolymer may improve the wettability of a film prepared by the composition according to the present invention.

According to one embodiment of the present invention, it is preferable that the (e) anionic polymer be selected from hyaluronic acid and derivatives thereof.

Hyaluronic acid can be represented by the following chemical formula.

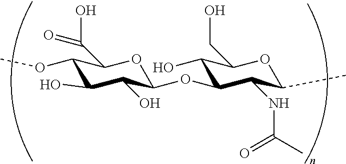

In the context of the present invention, the term “hyaluronic acid” covers in particular the basic unit of hyaluronic acid of formula:

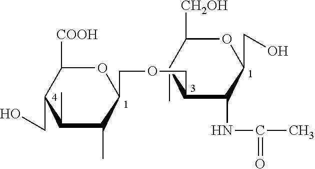

This is the smallest fraction of hyaluronic acid comprising a disaccharide dimer, namely D-glucuronic acid and N-acetylglucosamine.

The term “hyaluronic acid and derivatives thereof” also comprises, in the context of the present invention, the linear polymer comprising the polymeric unit described above, linked together in the chain via alternating p(1,4) and p(1,3) glycosidic linkages, having a molecular weight (MW) that can range between 380 and 13,000,000 daltons. This molecular weight depends in large part on the source from which the hyaluronic acid is obtained and/or on the preparation methods.

The term “hyaluronic acid and derivatives thereof” also comprises, in the context of the present invention, hyaluronic acid salts. As the salts, mention may be made of alkaline metal salts such as sodium salts and potassium salts, alkaline earth metal salts such as magnesium salts, ammonium salts, and mixtures thereof.

In the natural state, hyaluronic acid is present in pericellular gels, in the base substance of the connective tissues of vertebrate organs such as the dermis and epithelial tissues, and in particular in the epidermis, in the synovial fluid of the joints, in the vitreous humor, in the human umbilical cord and in the crista galli apophysis.

Thus, the term “hyaluronic acid and derivatives thereof” comprises all the fractions or subunits of hyaluronic acid having a molecular weight in particular within the molecular weight range recalled above.

In the context of the present invention, hyaluronic acid fractions, which do not have inflammatory activity, are preferably used.

By way of illustration of the various hyaluronic acid fractions, reference may be made to the document “Hyaluronan fragments: an information-rich system”, R. Stem et al., European Journal of Cell Biology 58 (2006) 699-715, which reviews the listed biological activities of hyaluronic acid according to its molecular weight.

According to a preferred embodiment of the present invention, the hyaluronic acid fractions suitable for the use covered by the present invention have a molecular weight of between 50,000 and 5,000,000, in particular between 100,000 and 5,000,000, especially between 400,000 and 5,000,000 Da. In this case, the term used is high-molecular-weight hyaluronic acid.

Alternatively, the hyaluronic acid fractions that may also be suitable for the use covered by the present invention have a molecular weight of between 50,000 and 400,000 Da. In this case, the term used is intermediate-molecular-weight hyaluronic acid.

Alternatively again, the hyaluronic acid fractions that may be suitable for the use covered by the present invention have a molecular weight of less than 50,000 Da. In this case, the term used is low-molecular-weight hyaluronic acid.

Finally, the term “hyaluronic acid and derivatives thereof” also comprises hyaluronic acid esters in particular those in which all or some of the carboxylic groups of the acid functions are esterified with oxyethylenated alkyls or alcohols, containing from 1 to 20 carbon atoms, in particular with a degree of substitution at the level of the D-glucuronic acid of the hyaluronic acid ranging from 0.5 to 50%.

Mention may in particular be made of methyl, ethyl, n-propyl, n-pentyl, benzyl and dodecyl esters of hyaluronic acid. Such esters have in particular been described in D. Campoccia et al. “Semisynthetic resorbable materials from hyaluronan esterification”, Biomaterials 19 (1998) 2101-2127.

The hyaluronic acid derivative may be, for example, acetylated hyaluronic acid or a salt thereof.

The molecular weights indicated above are also valid for the hyaluronic acid esters.

The hyaluronic acid may in particular be hyaluronic acid supplied by the company Hyactive under the trade name CPN (MW: 10 to 150 kDa), by the company Soliance under the trade name Cristalhyal (MW: 1.1.times.10⁶), by the company Bioland under the name Nutra HA (MW: 820 000 Da), by the company Bioland under the name Nutra AF (MW: 69 000 Da), by the company Bioland under the name Oligo HA (MW: 6100 Da) or else by the company Vam Farmacos Metica under the name D Factor (MW: 380 Da).

It may be preferable that the (e) anionic polymer be selected from natural anionic polymers, and more preferably from polysaccharides.

It may be even more preferable that the (e) anionic polymer be selected from the group consisting of hyaluronic acid and derivatives thereof, cellulose polymers and salts thereof such as carboxymethylcellulose and cellulose gum (sodium carboxymethylcellulose), and mixtures thereof.

Thus, the (e) anionic polymer may be selected from polysaccharides, preferably hyaluronic acid and derivatives thereof, cellulose polymers and salts thereof, and a mixture thereof, and more preferably hyaluronic acid and salts thereof (e.g., sodium hyaluronate), carboxymethylcellulose and salts thereof such as cellulose gum, and a mixture thereof.

The amount of the (e) anionic polymer(s) in the composition according to the present invention may be 0.01% by weight or more, preferably 0.05% by weight or more, and more preferably 0.1% by weight or more, relative to the total weight of the composition.

The amount of the (e) anionic polymer(s) in the composition according to the present invention may be 15% by weight or less, preferably 10% by weight or less, and more preferably 5% by weight or less, relative to the total weight of the composition.

The amount of the (e) anionic polymer(s) in the composition according to the present invention may be from 0.01% to 15% by weight, preferably from 0.05% to 10% by weight, and more preferably from 0.1% to 5% by weight, relative to the total weight of the composition.

The total amount of the (a) cationic polymer(s) and the (e) anionic polymer(s) in the composition according to the present invention may be 0.1% by weight or more, preferably 0.5% by weight or more, and more preferably 1% by weight or more, relative to the total weight of the composition.

The total amount of the (a) cationic polymer(s) and the (e) anionic polymer(s) in the composition according to the present invention may be 20% by weight or less, preferably 15% by weight or less, and more preferably 10% by weight or less, relative to the total weight of the composition.

The total amount of the (a) cationic polymer(s) and the (e) anionic polymer(s) in the composition according to the present invention may be from 0.1% to 20% by weight, preferably from 0.5% to 15% by weight, and more preferably from 1% to 10% by weight, relative to the total weight of the composition.

The ratio of the amount, for example the chemical equivalent, of the (a) cationic polymer(s)/the (e) anionic polymer(s) may be 0.05-18, preferably 0.1-10, and more preferably 0.5-5.0. In particular, it may be preferable that the number of cationic groups of the (a) cationic polymer(s)/the number of anionic groups of the (e) anionic polymer(s) be 0.05-18, more preferably 0.1-10, and even more preferably 0.5-5.0.

### (Non-Polymeric Acid Having Two or More Acid Dissociation Constants)

The composition according to the present invention includes (b) at least one non-polymeric acid having two or more pKa values or salt(s) thereof, i.e., at least one non-polymeric acid having two or more acid dissociation constants or salt(s) thereof. The pKa value (acid dissociation constant) is well known to those skilled in the art, and should be determined at a constant temperature such as 25° C.

The (b) non-polymeric acid having two or more pKa values can function as a crosslinker for the (a) cationic polymer. The (a) cationic polymer(s) can be ionically-crosslinked by the (b) non-polymeric acid(s) having two or more pKa values or salt(s) thereof.

The term “non-polymeric” here means that the acid is not obtained by polymerizing two or more monomers. Therefore, the non-polymeric acid does not correspond to an acid obtained by polymerizing two or more monomers such as polycarboxylic acid.

It is preferable that the molecular weight of the (b) non-polymeric acid having two or more pKa values or salt(s) thereof is 1,000 or less, preferably 800 or less, and more preferably 700 or less.

There is no limit to the type of the (b) non-polymeric acid having two or more pKa values or salt(s) thereof. Two or more different types of non-polymeric acids having two or more pKa values or salts thereof may be used in combination. Thus, a single type of a non-polymeric acid having two or more pKa values or a salt thereof or a combination of different types of non-polymeric acids having two or more pKa values or salts thereof may be used.

The term “salt” here means a salt formed by addition of suitable base(s) to the non-polymeric acid having two or more pKa values, which may be obtained from a reaction with the non-polymeric acid having two or more pKa values with the base(s) according to methods known to those skilled in the art. As the salt, mention may be made of metal salts, for example salts with alkaline metal such as Na and K, and salts with alkaline earth metal such as Mg and Ca, and ammonium salts.

The (b) non-polymeric acid having two or more pKa values or salt(s) thereof may be an organic acid or salt(s) thereof, and preferably a hydrophilic or water-soluble organic acid or salt(s) thereof.

The (b) non-polymeric acid having two or more pKa values may have at least two acid groups selected from the group consisting of a carboxylic group, a sulfuric group, a sulfonic group, a phosphoric group, a phosphonic group, a phenolic hydroxyl group, and a mixture thereof.

The (b) non-polymeric acid having two or more pKa values may be a non-polymeric polyvalent acid.

The (b) non-polymeric acid having two or more pKa values may be selected from the group consisting of dicarboxylic acids, disulfonic acids, and diphosphoric acids, and a mixture thereof.

The (b) non-polymeric acid having two or more pKa values or salt(s) thereof may be selected from the group consisting of oxalic acid, malonic acid, succinic acid, glutaric acid, adipic acid, pimelic acid, suberic acid, azelaic acid, sebacic acid, fumaric acid, maleic acid, malic acid, citric acid, aconitic acid, oxaloacetic acid, tartaric acid, and salts thereof; aspartic acid, glutamic acid, and salts thereof; terephthalylidene dicamphor sulfonic acid or salts thereof 50 (Mexoryl SX), Benzophenone-9; phytic acid, and salts thereof; Red 2 (Amaranth), Red 102 (New Coccine), Yellow 5 (Tartrazine), Yellow 6 (Sunset Yellow FCF), Green 3 (Fast Green FCF), Blue 1 (Brilliant Blue FCF), Blue 2 (Indigo Carmine), Red 201 (Lithol Rubine B), Red 202 (Lithol Rubine BCA), Red 204 (Lake Red CBA), Red 206 (Lithol Red CA), Red 207 (Lithol Red BA), Red 208 (Lithol Red SR), Red 219 (Brilliant Lake Red R), Red 220 (Deep Maroon), Red 227 (Fast Acid Magenta), Yellow 203 (Quinoline Yellow WS), Green 201 (Alizanine Cyanine Green F), Green 204 (Pyranine Conc), Green 205 (Light Green SF Yellowish), Blue 203 (Patent Blue CA), Blue 205 (Alfazurine FG), Red 401 (Violamine R), Red 405 (Permanent Re F5R), Red 502 (Ponceau 3R), Red 503 (Ponceau R), Red 504 (Ponceau SX), Green 401 (Naphthol Green B), Green 402 (Guinea Green B), and Black 401 (Naphthol Blue Black); folic acid, ascorbic acid, erythorbic acid, and salts thereof; cystine and salts thereof; EDTA and salts thereof; glycyrrhizin and salts thereof; and a mixture thereof.

It may be preferable that the (b) non-polymeric acid having two or more pKa values or salt(s) thereof be selected from the group consisting of terephthalylidene dicamphor sulfonic acid and salts thereof (Mexoryl SX), Yellow 6 (Sunset Yellow FCF), ascorbic acid, phytic acid and salts thereof, and a mixture thereof.

It may be more preferable that the (b) non-polymeric acid having two or more pKa values or salt(s) thereof be selected from the group consisting of terephthalylidene dicamphor sulfonic acid and salts thereof (Mexoryl SX), phytic acid and salts thereof, and a mixture thereof.

The amount of the (b) non-polymeric acid having two or more pKa values or salt(s) thereof in the composition according to the present invention may be 0.01% by weight or more, preferably 0.05% by weight or more, and more preferably 0.1% by weight or more, relative to the total weight of the composition.

The amount of the (b) non-polymeric acid having two or more pKa values or salt(s) thereof in the composition according to the present invention may be 15% by weight or less, preferably 10% by weight or less, and more preferably 5% by weight or less, relative to the total weight of the composition.

The amount of the (b) non-polymeric acid having two or more pKa values or salt(s) thereof in the composition according to the present invention may be from 0.01% to 15% by weight, preferably from 0.05% to 10% by weight, and more preferably from 0.1% to 5% by weight, relative to the total weight of the composition.

### [Polyol]

The composition according to the present invention includes (c) at least one polyol. If two or more polyols are used, they may be the same or different.

The term “polyol” here means an alcohol having two or more hydroxy groups, and does not encompass a saccharide or a derivative thereof. The derivative of a saccharide includes a sugar alcohol which is obtained by reducing one or more carbonyl groups of a saccharide, as well as a saccharide or a sugar alcohol in which the hydrogen atom or atoms in one or more hydroxy groups thereof has or have been replaced with at least one substituent such as an alkyl group, a hydroxyalkyl group, an alkoxy group, an acyl group or a carbonyl group.

Polyols used in the present invention are liquid at ambient temperature such as 25° C. under 50 atmospheric pressure (760 mmHg or 1.05 Pa).

The (c) polyol may be a C₂₋₂₄ polyol, preferably a C₂₋₉ polyol, comprising at least 2 hydroxy groups, and preferably 2 to 5 hydroxy groups.

The (c) polyol may be a natural or synthetic polyol. The polyol may have a linear, branched or cyclic molecular structure.

The (c) polyol may be selected from glycerins and derivatives thereof, and glycols and derivatives thereof. The polyol may be selected from the group consisting of glycerin, diglycerin, polyglycerin, ethyleneglycol, diethyleneglycol, propyleneglycol, dipropyleneglycol, butyleneglycol, pentyleneglycol, hexyleneglycol, C₆-C₂₄ polyethyleneglycol, 1,3-propanediol, 1,4-butanediol, and 1,5-pentanediol.

It is preferable that the (c) polyol be glycerin.

The amount of the (c) polyol(s) in the composition used in the present invention is 10% by weight or more, preferably 15% by weight or more, and more preferably 20% by weight or more, relative to the total weight of the composition.

On the other hand, the amount of the (c) polyol(s) in the composition used in the present invention may be 95% by weight or less, preferably 60% by weight or less, and more preferably 30% by weight or less, relative to the total weight of the composition.

The amount of the (c) polyol(s) in the composition used in the present invention may range from 10% to 95% by weight, preferably from 10% to 60% by weight, and more preferably from 10% to 30% by weight, relative to the total weight of the composition.

### [Water]

The composition according to the present invention includes (d) water.

The amount of the (d) water may be 1% by weight or more, preferably 30% by weight or more, and more preferably 50% by weight or more, relative to the total weight of the composition.

The amount of the (d) water may be 90% by weight or less, preferably 50% by weight or less, and more preferably 10% by weight or less, relative to the total weight of the composition.

The amount of the (d) water may be from 1% to 90% by weight, preferably from 30% to 90% by weight, and more preferably from 50% to 90% by weight, relative to the total weight of the composition.

[pH]

The pH of the composition according to the present invention may be 9.0 or less, preferably 8.5 or less, and more preferably 8.1 or less.

The pH of the composition according to the present invention may be 3.0 or more, preferably 3.3 or more, and more preferably 3.5 or more.

The pH of the composition according to the present invention may be from 3.0 to 9.0, preferably from 3.3 to 8.5, and more preferably from 3.5 to 8.1.

At a pH of from 3.0 to 9.0, the composition according to the present invention can be very stable.

The pH of the composition according to the present invention may be adjusted by adding at least one alkaline agent and/or at least one acid, other than the (b) non-polymeric acid having two or more pKa values or salt(s) thereof. The pH of the composition according to the present invention may also be adjusted by adding at least one buffering agent.

### (Alkaline Agent)

The composition according to the present invention may comprise at least one alkaline agent. Two or more alkaline agents may be used in combination. Thus, a single type of alkaline agent or a combination of different types of alkaline agents may be used.

The alkaline agent may be an inorganic alkaline agent. It is preferable that the inorganic alkaline agent be selected from the group consisting of ammonia; alkaline metal hydroxides; alkaline earth metal hydroxides; alkaline metal phosphates and monohydrogenophosphates such as sodium phosphate or sodium monohydrogen phosphate.

As examples of the inorganic alkaline metal hydroxides, mention may be made of sodium hydroxide and potassium hydroxide. As examples of the alkaline earth metal hydroxides, mention may be made of calcium hydroxide and magnesium hydroxide. As an inorganic alkaline agent, sodium hydroxide is preferable.

The alkaline agent may be an organic alkaline agent. It is preferable that the organic alkaline agent be selected from the group consisting of monoamines and derivatives thereof; diamines and derivatives thereof; polyamines and derivatives thereof; basic amino acids and derivatives thereof; oligomers of basic amino acids and derivatives thereof; polymers of basic amino acids and derivatives thereof; urea and derivatives thereof; and guanidine and derivatives thereof.

As examples of the organic alkaline agents, mention may be made of alkanolamines such as mono-, di- and tri-ethanolamine, and isopropanolamine; urea, guanidine and their derivatives; basic amino acids such as lysine, ornithine or arginine; and diamines such as those described in the structure below:

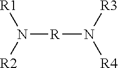

wherein R denotes an alkylene such as propylene optionally substituted by a hydroxyl or a C₁-C₄ alkyl radical, and R₁, R₂, R₃ and R₄ independently denote a hydrogen atom, an alkyl radical or a C₁-C₄ hydroxyalkyl radical, which may be exemplified by 1,3-propanediamine and derivatives thereof. Arginine, urea and monoethanolamine are preferable.

The alkaline agent(s) may be used in a total amount of from 0.01% to 15% by weight, preferably from 0.02% to 10% by weight, more preferably from 0.03% to 5% by weight, relative to the total weight of the composition, depending on their solubility.

### (Acid)

The composition according to the present invention may comprise at least one acid. Two or more acids may be used in combination. Thus, a single type of acid or a combination of different types of acids may be used.

As the acid, mention may be made of any inorganic or organic acids, preferably inorganic acids, which are commonly used in cosmetic products. A monovalent acid and/or a polyvalent acid may be used. A monovalent acid such as citric acid, lactic acid, sulfuric acid, phosphoric acid and hydrochloric acid (HCl) may be used. HCl is preferable.

The acid(s) may be used in a total amount of from 0.01% to 15% by weight, preferably from 0.02% to 10% by weight, more preferably from 0.03% to 5% by weight, relative to the total weight of the composition, depending on their solubility.

### (Buffering Agent)

The composition according to the present invention may comprise at least one buffering agent. Two or more buffering agents may be used in combination. Thus, a single type of buffering agent or a combination of different types of buffering agents may be used.

As the buffering agent, mention may be made of an acetate buffer (for example, acetic acid+sodium acetate), a phosphate buffer (for example, sodium dihydrogen phosphate+di-sodium hydrogen phosphate), a citrate buffer (for example, citric acid+sodium citrate), a borate buffer (for example, boric acid+sodium borate), a tartrate buffer (for example, tartaric acid+sodium tartrate dihydrate), Tris buffer (for example, tris(hydroxymethyl)aminomethane), and a Hepes buffer (4-(2-hydroxyethyl)-1-piperazineethanesulfonic acid).

### [Optional Ingredients]

The composition according to the present invention may comprise, in addition to the aforementioned essential ingredients (ingredients (a) to (d)), optional ingredients such as the (e) anionic polymer and those typically employed in cosmetics, specifically, surfactants (in particular, nonionic surfactants) or emulsifiers, hydrophilic or lipophilic thickeners, organic volatile or non-volatile solvents other than the ingredient (c), hydrophilic or hydrophobic UV filters, silicones and silicone derivatives, natural extracts derived from animals or vegetables, oils, waxes, and the like, within a range which does not impair the effects of the present invention.

The composition according to the present invention may comprise the above optional ingredient(s) in an amount of from 0.01% to 50% by weight, preferably from 0.05% to 30% by weight, and more preferably from 0.1% to 10% by weight, relative to the total weight of the composition.

The composition according to the present invention may include no surfactant or include a limited amount of surfactant, e.g., less than 0.1% by weight, preferably less than 0.01% by weight, and more preferably less than 0.001% by weight, relative to the total weight of the composition.

### [Preparation]

The composition according to the present invention can be prepared by mixing the above-described essential and optional ingredients in a conventional manner.

For example, the composition according to the present invention can be prepared by a process comprising the step of mixing
(a) at least one cationic polymer,
(b) at least one non-polymeric acid having two or more pKa values or salt(s) thereof,
(c) at least one polyol, and
(d) water,
wherein
the amount of the (c) polyol(s) in the composition is 10% by weight or more, relative to the total weight of the composition.

It is possible to further mix any of the optional ingredients. For example, the (e) at least one anionic polymer may be mixed.

The mixing can be performed at any temperature such as room temperature (e.g., 25° C.), preferably without heating. The composition according to the present invention can be environmentally friendly because it can be prepared without any heating step.

It is preferable that the cosmetic composition according to the present invention be in the form of a liquid at a room temperature (e.g., 25° C.).

### [Film]

The composition according to the present invention may be used for easily preparing a film.

Thus, the present invention may also relate to a process for preparing a film, preferably a cosmetic film, optionally with a thickness of preferably 10 nm or more, more preferably 50 nm or more, and even more preferably 100 nm or more, comprising:
applying onto a substrate, preferably a keratin substance, and more preferably skin and hair, the composition according to the present invention; and
drying the composition.

The upper limit of the thickness of the above film is not limited. Thus, for example, the thickness of the above film may be 10 μm or less, preferably 5 μm or less, more preferably 3 μm or less, and even more preferably 1 μm or less.

Thus, the present invention may also relate to a film, preferably a cosmetic film, comprising:
(a) at least one cationic polymer;
(b) at least one non-polymeric acid having two or more pKa values or salt(s) thereof; and
(c) at least one polyol.

The film may further comprise (d) water.

The above film, preferably the cosmetic film, is resistant to water with a pH of 7 or less, and is removable with water with a pH of more than 7, preferably 8 or more, and more preferably 9 or more.

In other words, the above film, preferably the cosmetic film, can be water-resistant under neutral or acidic conditions such as a pH of 7 or less, preferably in a range of 6 or more and 7 or less, and more preferably in a range of 5 or more and 7 or less, while the above film preferably the cosmetic film, can be removed under alkaline conditions such as a pH of more than 7, preferably 8 or more, and more preferably 9 or more. The upper limit of the pH is preferably 13, more preferably 12, and even more preferably 11.

Accordingly, the above film, preferably the cosmetic film, can be water-resistant, and therefore, it can remain on a keratin substance such as skin and hair even if the surface of the keratin substance is wet due to, for example sweat and rain. On the other hand, the above film, preferably the cosmetic film, can be easily removed from the keratin substance under alkaline conditions. Therefore, the above film, preferably the cosmetic film, is difficult to remove with water, while it can be easily removed with, for example, a soap which can provide alkaline conditions.

Furthermore, the above film may have cosmetic effects such as absorbing or adsorbing malodor, changing the appearance of a keratin substance such as skin and hair, changing the feel to the touch of the keratin substance, and/or protecting the keratin substance from, for example, dirt or pollutant, due to the properties of the film, even if the film does not include any cosmetic active ingredient.

As the above film includes (c) at least one polyol such as glycerin, the film can have cosmetic effects provided by the (c) polyol, e.g., moisturizing effects.

### [Cosmetic Use and Process]

The composition according to the present invention may be used as a cosmetic composition, preferably a care cosmetic composition, and more preferably a skin care or hair care cosmetic composition.

Thus, the cosmetic composition according to the present invention may be intended for application onto a keratin substance. The keratin substance may be keratin fibers. Keratin fibers here mean fibers containing keratin as a main constituent element, and examples thereof include hair, eyelash, eyebrow, and the like. On the other hand, the keratin substance may be skin and mucous membranes such as lips.

The composition according to the present invention can preferably be used as a leave-on or rinse-off cosmetic composition for a keratin substance such as skin and hair.

The “leave-on” here means that the composition according to the present invention is not removed from a keratin substance such as skin and hair after being applied onto the keratin substance.

The “rinse-off” here means that the composition according to the present invention is removed, by rinsing, from a keratin substance such as skin and hair after being applied onto the keratin substance. Water can be used for rinsing. Even after rinsing off, the composition according to the present invention can remain to form a film or a coating on a keratin substance such as skin and hair.

The present invention also relates to
a cosmetic process for a keratin substance such as skin and hair, comprising:
applying to the keratin substance the composition according to the present invention; and
drying the composition to form a cosmetic film on the keratin substance, and
a process for preparing a film, preferably a cosmetic film, comprising:
applying onto a keratin substance such as skin and hair, the composition according to the present invention; and
drying the composition.

The cosmetic process here means a non-therapeutic cosmetic method for caring a keratin substance such as skin and hair, and/or styling keratin fibers such as hair.

The present invention may also relate to a use of the composition according to the present invention for the preparation of a cosmetic film on a keratin substance such as skin and hair.

The present invention may also relate to a use of:
(a) at least one cationic polymer; and
(b) at least one non-polymeric acid having two or more pKa values or salt(s) thereof, in a cosmetic composition for a keratin substance such as skin and hair, comprising:
(c) at least one polyol; and
(d) water,
wherein
the amount of the (c) polyol(s) in the composition is 10% by weight or more, relative to the total weight of the composition,
in order to reduce stickiness of the composition.

The use according to the present invention can reduce the stickiness of a composition including the ingredients (c) and (d) wherein the amount of the ingredient (c) in the composition is 10% by weight or more, relative to the total weight of the composition, by a combination of the ingredients (a) and (b), optionally with the ingredient (e).

The explanations regarding the ingredients (a) to (e) in the composition according to the present invention can apply to those in the use according to the present invention.

### EXAMPLES

The present invention will be described in a more detailed manner by way of examples.

However, they should not be construed as limiting the scope of the present invention.

### Examples 1-4 and Comparative Examples 1-4

### [Preparation]

Each of the compositions according to Examples 1-4 and Comparative Examples 1-4 was 50 prepared by mixing the ingredients shown in Table 1 at a room temperature (25° C.). The numerical values for the amounts of the ingredients in Table 1 are all based on “% by weight” as raw materials.

TABLE 1

| | | | | | Comp. | Comp. | Comp. | Comp. |
|---|---|---|---|---|---|---|---|---|
| | Ex. 1 | Ex. 2 | Ex. 3 | Ex. 4 | Ex. 1 | Ex. 2 | Ex. 3 | Ex. 4 |
| Polylysine | 2 | 2 | — | — | 2 | 2 | — | — |
| Chitosan | — | — | 2 | 2 | — | — | 2 | 2 |
| Phytic Acid | 0.55 | — | 0.55 | 0 | — | 0.55 | — | 0.55 |
| Terephthalylidene | — | 1 | 0 | 0.55 | — | — | — | — |
| Dicamphor | | | | | | | | |
| Sulfonic Acid | | | | | | | | |
| Glycerin | 10 | 10 | 10 | 10 | 10 | — | 10 | — |
| Water | qsp 100 | qsp 100 | qsp 100 | qsp 100 | qsp 100 | qsp 100 | qsp 100 | qsp 100 |
| Formation of | Good | Good | Fair | Fair | Very | Very | Very | Very |
| Gel Film | | | | | Poor | Poor | Poor | Poor |
| Texture | Very | Very | Good | Good | Very | NA | Very | NA |
| | Good | Good | | | Poor | | Poor | |

### [Evaluations]

### (Formation of Gel Film)

Each of the compositions according to Examples 1-4 and Comparative Examples 1-4, in an amount of 10 g, was poured onto a Petri dish, and dried at 45° C. for 24 hours.

Five experts evaluated the formation of gel film in the Petri dish in accordance with the following grade:
5: Homogeneous gel film was formed (gel film was presented evenly on the Petri dish)
4: Gel film was formed, but it was not homogeneous (gel film was presented unevenly on the Petri dish)
3: Gel film was formed slightly
2: Gel film was formed very slightly
1: Gel film was not formed

It was then classified in the following categories based on the average of the grade.
5: Very Good
4: Good
3: Fair
2: Poor
1: Very Poor

The results are shown in Table 1.

### (Texture)

Each of the compositions according to Examples 1-4 and Comparative Examples 1-4, in an amount of 10 g, was poured onto a Petri dish, and dried at 45° C. for 24 hours.

Five experts evaluated the texture on the Petri dish in accordance with the following grade:
5: Not sticky (Very smooth)
4: Very slightly sticky (Smooth)
3: Slightly sticky
2: Sticky
1: Very sticky

It was then classified in the following categories based on the average of the grade.
5: Very Good
4: Good
3: Fair
2: Poor
1: Very Poor

The results are shown in Table 1.

### (Summary)

The compositions according to Examples 1-4 formed a gel film. The compositions according to Examples 1 and 2 formed a gel film better than the compositions according to Examples 3 and 4.

The compositions according to Comparative Examples 1-4 could not form any gel.

As the compositions according to Comparative Examples 2 and 4 did not include glycerin, the texture of the compositions was not evaluated.

It is demonstrated that a cationic polymer (polylysine and chitosan), a crosslinker (phytic acid or terephthalylidene dicamphor sulfonic acid) and glycerin can form a gel film (coacervate) and can reduce the stickiness which is inherent to glycerin.

### Examples 5-6 and Comparative Examples 5-6

### [Preparation]

Each of the compositions according to Examples 5-6 and Comparative Examples 5-6 was prepared by mixing the ingredients shown in Table 2 at room temperature (25° C.). The numerical values for the amounts of the ingredients in Table 2 are all based on “% by weight” as raw materials.

TABLE 2

| | | | Comp. | Comp. |
|---|---|---|---|---|
| | Ex. 5 | Ex. 6 | Ex. 5 | Ex. 6 |
| Polylysine | 2 | 2 | 2 | 2 |
| Sodium Hyaluronate | 1 | — | — | — |
| Cellulose Gum | — | 1 | — | 1 |
| Phytic Acid | 0.55 | 0.55 | 0.55 | — |
| Glycerin | 10 | 10 | — | 10 |
| Water | qsp 100 | qsp 100 | qsp 100 | qsp 100 |
| pH | 6.9 | 7.4 | 6.5 | 9.8 |
| Formation of Gel Film | Very | Very | Very | Very |
| | Good | Good | Poor | Poor |
| Texture | Very | Very | NA | Very |
| | Good | Good | | Poor |

### [Evaluations]

### (Formation of Gel Film)

Each of the compositions according to Examples 5-6, and Comparative Examples 5-6, in an amount of 10 g, was poured onto a Petri dish, and dried at 45° C. for 24 hours.

Five experts evaluated the formation of gel film in the Petri dish in accordance with the following grade:
5: Homogeneous gel film was formed (gel film was presented evenly on the Petri dish)
4: Gel film was formed, but it was not homogeneous (gel film was presented unevenly on the Petri dish)
3: Gel film was formed slightly
2: Gel film was formed very slightly
1: Gel film was not formed

It was then classified in the following categories based on the average of the grade.
5: Very Good
4: Good
3: Fair
2: Poor
1: Very Poor

The results are shown in Table 2.

### (Texture)

Each of the compositions according to Examples 5-6, and Comparative Examples 5-6, in an amount of 10 g, was poured onto a Petri dish, and dried at 45° C. for 24 hours.

Five experts evaluated the texture on the Petri dish in accordance with the following grade:
5: Not sticky (Very smooth)
4: Very slightly sticky (Smooth)
3: Slightly sticky
2: Sticky
1: Very sticky

It was then classified in the following categories based on the average of the grade.
5: Very Good
4: Good
3: Fair
2: Poor
1: Very Poor

The results are shown in Table 2.

### (Summary)

The compositions according to Examples 5-6 formed a homogeneous gel film. The gel film was spread on the entirety of the surface of the Petri dish.

The compositions according to Comparative Examples 5-6 could not form any gel.

As the composition according to Comparative Example 5 did not include glycerin, the stickiness of the composition was not evaluated.

It is demonstrated that an anionic polymer (sodium hyaluronate and cellulose gum) can be added to a cationic polymer (polylysine), a crosslinker (phytic acid) and glycerin to form a gel film (coacervate) and that the gel film thus formed is more preferable (more homogeneous).

### Examples 7-8 and Comparative Examples 7-8

### [Preparation]

Each of the compositions according to Examples 7-8 and Comparative Examples 7-8 was prepared by mixing the ingredients shown in Table 3 at room temperature (25° C.). The numerical values for the amounts of the ingredients in Table 3 are all based on “% by weight” as raw materials.

TABLE 3

| | | | Comp. | Comp. |
|---|---|---|---|---|
| | Ex. 7 | Ex. 8 | Ex. 7 | Ex. 8 |
| Polylysine | 2 | 2 | 2 | — |
| Phytic Acid | 0.55 | 0.55 | — | — |
| Glycerin | 50 | 90 | 50 | 50 |
| Water | qsp 100 | qsp 100 | qsp 100 | qsp 100 |
| pH | 6.5 | 6.5 | 9.7 | 5.7 |
| Formation of Gel Film | Good | Good | Very | Very |
| | | | Poor | Poor |
| Texture | Very | Very | Very | Very |
| | Good | Good | Poor | Poor |

### [Evaluations]

### (Formation of Gel Film)

Each of the compositions according to Examples 7-8, and Comparative Examples 7-8, in an amount of 10 g, was poured onto a Petri dish, and dried at 45° C. for 24 hours.

Five experts evaluated the formation of gel film in the Petri dish in accordance with the following grade:
5: Homogeneous gel film was formed (gel film was presented evenly on the Petri dish)
4: Gel film was formed, but it was not homogeneous (gel film was presented unevenly on the Petri dish)
3: Gel film was formed slightly
2: Gel film was formed very slightly
1: Gel film was not formed

It was then classified in the following categories based on the average of the grade.
5: Very Good
4: Good
3: Fair
2: Poor
1: Very Poor

The results are shown in Table 3.

### (Texture)

Each of the compositions according to Examples 7-8, and Comparative Examples 7-8, in an amount of 10 g, was poured onto a Petri dish, and dried at 45° C. for 24 hours.

Five experts evaluated the texture on the Petri dish in accordance with the following grade:
5: Not sticky (Very smooth)
4: Very slightly sticky (Smooth)
3: Slightly sticky
2: Sticky
1: Very sticky

It was then classified in the following categories based on the average of the grade.
5: Very Good
4: Good
3: Fair
2: Poor
1: Very Poor

The results are shown in Table 3.

### (Summary)

The compositions according to Examples 7-8 formed a gel.

The compositions according to Comparative Examples 7-8 could not form any gel.

It is demonstrated that a large amount of glycerin can be used to form a gel film (coacervate).

### Example 9 and Comparative Example 9

### [Preparation]

Each of the compositions according to Example 9 and Comparative Example 9 was prepared by mixing the ingredients shown in Table 4 at room temperature (25° C.). The numerical values for the amounts of the ingredients in Table 4 are all based on “% by weight” as raw materials.

TABLE 4

| | | Comp. |
|---|---|---|
| | Ex. 9 | Ex. 9 |
| Polylysine | 4 | — |
| Phytic Acid | 1.1 | — |
| Glycerin | 10 | 10 |
| Water | qsp 100 | qsp 100 |
| pH | 6.7 | 6.3 |
| Formation of Gel Film | Good | Very |
| | | Poor |
| Texture | Very | Very |
| | Good | Poor |

### [Evaluations]

### (Formation of Gel Film)

Each of the compositions according to Example 9 and Comparative Example 9, in an amount of 10 g, was poured onto a Petri dish, and dried at 45° C. for 24 hours.

Five experts evaluated the formation of gel film in the Petri dish in accordance with the following grade:
5: Homogeneous gel film was formed (gel film was presented evenly on the Petri dish)
4: Gel film was formed, but it was not homogeneous (gel film was presented unevenly on the Petri dish)
3: Gel film was formed slightly
2: Gel film was formed very slightly
1: Gel film was not formed

It was then classified in the following categories based on the average of the grade.
5: Very Good
4: Good
3: Fair
2: Poor
1: Very Poor

The results are shown in Table 4.

### (Texture)

Each of the compositions according to Example 9 and Comparative Example 9, in an amount of 10 g, was poured onto a Petri dish, and dried at 45° C. for 24 hours.

Five experts evaluated the texture on the Petri dish in accordance with the following grade:
5: Not sticky (Very smooth)
4: Very slightly sticky (Smooth)
3: Slightly sticky
2: Sticky
1: Very sticky

It was then classified in the following categories based on the average of the grade.
5: Very Good
4: Good
3: Fair
2: Poor
1: Very Poor

The results are shown in Table 4.

### (Summary)

The composition according to Example 9 formed a gel.

The composition according to Comparative Example 9 could not form any gel.

It is demonstrated that a large amount of a cationic polymer and/or a large amount of a crosslinker can be used to form a gel film (coacervate).

### Examples 10-16

### [Preparation]

Each of the compositions according to Examples 10-16 was prepared by mixing the ingredients shown in Table 5 at room temperature (25° C.). The numerical values for the amounts of the ingredients in Table 5 are all based on “% by weight” as raw materials.

TABLE 5

| | Ex. 10 | Ex. 11 | Ex. 12 | Ex. 13 | Ex. 14 | Ex. 15 | Ex. 16 |
|---|---|---|---|---|---|---|---|
| Polylysine | 2 | 2 | 2 | 2 | 2 | 2 | 2 |
| Phytic Acid | 0.55 | 0.60 | 0.68 | 0.73 | 0.78 | 0.90 | 1.00 |
| Glycerin | 10 | 10 | 10 | 10 | 10 | 10 | 10 |
| Water | qsp 100 | qsp 100 | qsp 100 | qsp 100 | qsp 100 | qsp 100 | qsp 100 |
| pH | 6.5 | 6.14 | 5.07 | 4.50 | 4.05 | 3.05 | 2.58 |
| Formation of | Good | Good | Good | Good | Good | Good | Good |
| Gel Film | | | | | | | |
| Texture | Very | Very | Very | Very | Very | Very | Very |
| | Good | Good | Good | Good | Good | Good | Good |

### [Evaluations]

(Formation of Gel Film) Each of the compositions according to Examples 10-16, in an amount of 10 g, was poured onto a Petri dish, and dried at 45° C. for 24 hours.

Five experts evaluated the formation of gel film in the Petri dish in accordance with the following grade:
5: Homogeneous gel film was formed (gel film was presented evenly on the Petri dish)
4: Gel film was formed, but it was not homogeneous (gel film was presented unevenly on the Petri dish)
3: Gel film was formed slightly
2: Gel film was formed very slightly
1: Gel film was not formed

It was then classified in the following categories based on the average of the grade.
5: Very Good
4: Good
3: Fair
2: Poor
1: Very Poor

The results are shown in Table 5.

### (Texture)

Each of the compositions according to Examples 10-16, in an amount of 10 g, was poured onto a Petri dish, and dried at 45° C. for 24 hours.

Five experts evaluated the texture on the Petri dish in accordance with the following grade:
5: Not sticky (Very smooth)
4: Very slightly sticky (Smooth)
3: Slightly sticky
2: Sticky
1: Very sticky

It was then classified in the following categories based on the average of the grade.
5: Very Good
4: Good
3: Fair
2: Poor
1: Very Poor

The results are shown in Table 5.

### (Summary)

The compositions according to Examples 10-16 formed a gel. It is demonstrated that the amount of a crosslinker can vary to form a gel film (coacervate).

### Example 17 and Comparative Examples 10-14]

### [Preparation]

Each of the compositions according to Example 17 and Comparative Examples 10-14 was prepared by mixing the ingredients shown in Table 6 at room temperature (25° C.). The numerical values for the amounts of the ingredients in Table 6 are all based on “% by weight” as raw materials.

TABLE 6

| | | Comp. | Comp. | Comp. | Comp. | Comp. |
|---|---|---|---|---|---|---|
| | Ex. 17 | Ex. 10 | Ex. 11 | Ex. 12 | Ex. 13 | Ex. 14 |
| Polylysine | 2 | 2 | 2 | 2 | 2 | 2 |
| Phytic Acid | 0.55 | — | — | — | — | — |
| 0.1M HCl | — | 2.80 | 3.40 | 3.90 | 3.96 | 4.10 |
| Glycerin | 10 | 10 | 10 | 10 | 10 | 10 |
| Water | qsp 100 | qsp 100 | qsp 100 | qsp 100 | qsp 100 | qsp 100 |
| pH | 6.5 | 6.57 | 6.00 | 5.07 | 3.86 | 2.51 |
| Formation | Good | Very | Very | Very | Very | Very |
| of Gel Film | | Poor | Poor | Poor | Poor | Poor |
| Texture | Very | Very | Very | Very | Very | Very |
| | Good | Poor | Poor | Poor | Poor | Poor |

### [Evaluations]

### (Formation of Gel Film)

Each of the compositions according to Example 17 and Examples 10-16, in an amount of 10 g, was poured onto a Petri dish, and dried at 45° C. for 24 hours.

Five experts evaluated the formation of gel film in the Petri dish in accordance with the following grade:
5: Homogeneous gel film was formed (gel film was presented evenly on the Petri dish)
4: Gel film was formed, but it was not homogeneous (gel film was presented unevenly on the Petri dish)
3: Gel film was formed slightly
2: Gel film was formed very slightly
1: Gel film was not formed

It was then classified in the following categories based on the average of the grade.
5: Very Good
4: Good
3: Fair
2: Poor
1: Very Poor

The results are shown in Table 6.

### (Texture)

Each of the compositions according to Example 17 and Examples 10-16, in an amount of 10 g, was poured onto a Petri dish, and dried at 45° C. for 24 hours.

Five experts evaluated the texture on the Petri dish in accordance with the following grade:
5: Not sticky (Very smooth)
4: Very slightly sticky (Smooth)
3: Slightly sticky
2: Sticky
1: Very sticky

It was then classified in the following categories based on the average of the grade.
5: Very Good
4: Good
3: Fair
2: Poor
1: Very Poor

The results are shown in Table 6.

### (Summary)

The composition according to Example 17 formed a gel. The compositions according to Comparative Examples 10-14 could not form any gel.

It is demonstrated that the use of a mono-valent acid (HCl) could not form a gel film (coacervate). It can be understood that the use of a multi-valent crosslinker (for example, phytic acid) is necessary.

### Examples 18-23

### [Preparation]

Each of the compositions according to Examples 18-23 was prepared by mixing the ingredients shown in Table 7 at room temperature (25° C.). The numerical values for the amounts of the ingredients in Table 7 are all based on “% by weight” as raw materials.

TABLE 7

| | Ex. 18 | Ex. 19 | Ex. 20 | Ex. 21 | Ex. 22 | Ex. 23 |
|---|---|---|---|---|---|---|
| Polylysine | 2 | 2 | 2 | 2 | 2 | 2 |
| Terephthalylidene | 1 | 1.25 | 1.5 | 2.0 | 2.5 | 3.0 |
| Dicamphor | | | | | | |
| Sulfonic Acid | | | | | | |
| Glycerin | 10 | 10 | 10 | 10 | 10 | 10 |
| Water | qsp 100 | qsp 100 | qsp 100 | qsp 100 | qsp 100 | qsp 100 |
| pH | 8.1 | 8.0 | 7.9 | 7.6 | 7.3 | 5.7 |
| Formation of | Good | Good | Good | Good | Good | Good |
| Gel Film | | | | | | |
| Texture | Very | Very | Very | Very | Very | Very |
| | Good | Good | Good | Good | Good | Good |

### [Evaluations]

### (Formation of Gel Film)

Each of the compositions according to Examples 18-23, in an amount of 10 g, was poured onto a Petri dish, and dried at 45° C. for 24 hours.

Five experts evaluated the formation of gel film in the Petri dish in accordance with the following grade:
5: Homogeneous gel film was formed (gel film was presented evenly on the Petri dish)
4: Gel film was formed, but it was not homogeneous (gel film was presented unevenly on the Petri dish)
3: Gel film was formed slightly
2: Gel film was formed very slightly
1: Gel film was not formed

It was then classified in the following categories based on the average of the grade.
5: Very Good
4: Good
3: Fair
2: Poor
1: Very Poor

The results are shown in Table 7.

### (Texture)

Each of the compositions according to Examples 18-23, in an amount of 10 g, was poured onto a Petri dish, and dried at 45° C. for 24 hours.

Five experts evaluated the texture on the Petri dish in accordance with the following grade:
5: Not sticky (Very smooth)
4: Very slightly sticky (Smooth)
3: Slightly sticky
2: Sticky
1: Very sticky

It was then classified in the following categories based on the average of the grade.
5: Very Good
4: Good
3: Fair
2: Poor
1: Very Poor

The results are shown in Table 7.

### (Summary)

The compositions according to Examples 18-23 formed a gel.

It is demonstrated that a gel film can be formed at a pH of, at least, below 8.1.

1. A composition, comprising:
(a) at least one cationic polymer,
(b) at least one non-polymeric acid having two or more pKa values or salt(s) thereof;
(c) at least one polyol; and
(d) water,
wherein
the amount of the (c) polyol(s) in the composition is 10% by weight or more, relative to the total weight of the composition.

2. The composition according to claim 1, wherein the (a) cationic polymer is crosslinked with the (b) non-polymeric acid having two or more pKa values or salt(s) thereof.

3. The composition according to claim 1, wherein the (a) cationic polymer has at least one positively chargeable and/or positively charged moiety selected from the group consisting of a primary, secondary or tertiary amino group, a quaternary ammonium group, a guanidine group, a biguanide group, an imidazole group, an imino group, and a pyridyl group.

4. The composition according to claim 1, wherein the (a) cationic polymer is selected from the group consisting of cyclopolymers of alkyldiallylamine and cyclopolymers of dialkyldiallylammonium such as (co)polydiallyldialkyl ammonium chloride, (co)polyamines such as chitosans and (co)polylysines, cationic (co)polyaminoacids such as collagen, cationic cellulose polymers, and salts thereof.

5. The composition according to claim 1, wherein the (a) cationic polymer is selected from the group consisting of polylysines, chitosans, and mixtures thereof.

6. The composition according to claim 1, wherein the amount of the (a) cationic polymer(s) in the composition is from 0.01% to 15% by weight, preferably from 0.05% to 10% by weight, and more preferably from 0.1% to 5% by weight, relative to the total weight of the composition.

7. The composition according to claim 1, wherein the (b) non-polymeric acid having two or more pKa values or salt(s) thereof is an organic acid or salt(s) thereof, preferably a hydrophilic or water-soluble organic acid or salt(s) thereof, and more preferably phytic acid or salts thereof, terephthalylidene dicamphor sulfonic acid or salts thereof, or a mixture thereof.

8. The composition according to claim 1, wherein the amount of the (b) non-polymeric acid having two or more pKa values or salt(s) thereof in the composition is from 0.01% to 15% by weight, preferably from 0.05% to 10% by weight, and more preferably from 0.1% to 5% by weight, relative to the total weight of the composition.

9. The composition according to claim 1, wherein the (c) polyol is glycerin.

10. The composition according to claim 1, wherein the amount of the (c) polyol in the composition is 95% by weight or less, relative to the total weight of the composition.

11. The composition according to claim 1, wherein the composition further comprises (e) at least one anionic polymer.

12. The composition according to claim 11, wherein the (e) anionic polymer is selected from polysaccharides, preferably hyaluronic acid and derivatives thereof, cellulose polymers and salts thereof, and a mixture thereof, and more preferably hyaluronic acid and salts thereof, carboxymethylcellulose and salts thereof, and a mixture thereof.

13. The composition according to claim 11, wherein the amount of the (e) anionic polymer(s) in the composition is from 0.01% to 15% by weight, preferably from 0.05% to 10% by weight, and more preferably from 0.1% to 5% by weight, relative to the total weight of the composition.

14. The composition according to claim 1, wherein the composition is a cosmetic composition, preferably a care cosmetic composition, and more preferably a skin care or hair care cosmetic composition.

15. A cosmetic process for keratin substance, comprising
applying to the keratin substance the composition according to claim 1; and
drying the composition to form a cosmetic film on the keratin substance.
